(19) United States
(12) Patent Application Publication
(10) Pub. No.: US 2026/0108294 A1
(43) Pub. Date: Apr. 23, 2026

(54) MEDICAL DEVICE

(71) Applicants: TERUMO KABUSHIKI KAISHA, Tokyo (JP); TERUMO CARDIOVASCULAR SYSTEMS CORPORATION, Ann Arbor, MI (US)

(72) Inventors: Tatsunori Fujii, Kanagawa (JP); Yusaku Yamaki, Kanagawa (JP)

(21) Appl. No.: 19/394,055

(22) Filed: Nov. 19, 2025

Related U.S. Application Data
(63) Continuation of application No. PCT/JP2024/020846, filed on Jun. 7, 2024.

(30) Foreign Application Priority Data
Jun. 9, 2023 (JP) 2023-095458

(51) Int. Cl. A61B 18/14; A61B 17/29; A61B 18/00

(52) U.S. Cl. CPC A61B 18/1447; A61B 2017/2936; A61B 2017/2947; A61B 2018/00178

(57) ABSTRACT

A medical device includes a first jaw and a second jaw that pinch a piece of biological tissue. The second jaw is rotatable. The first jaw includes a first proximal portion, a first electrode, and a first power line electrically coupled to the first electrode. The first proximal portion includes a first inner surface and a first outer surface. The first inner surface is a surface facing a second proximal portion of the second jaw when the first jaw and the second jaw are in a closed state. The first outer surface is a back surface of the first inner surface. At least a part of the first power line is disposed along the first outer surface.

### CROSS-REFERENCE TO RELATED APPLICATIONS

Pursuant to 35 U.S. C. § 119(e), this application is a continuation of International Application No. PCT/JP2024/020846 with an international filing date of Jun. 7, 2024, which claims the benefit of Japanese Patent Application No. JP2023095458 filed on Jun. 9, 2023, the contents of which are hereby incorporated by reference in their entireties.

### TECHNICAL FIELD

The present invention relates to a medical device for pinching a piece of biological tissue between a first jaw and a second jaw that are openable and closable, when both the jaws are brought in a closed state.

### BACKGROUND

In coronary artery bypass surgery, a blood vessel bypassing a lesion site is coupled to an artery. The blood vessel is collected by an endoscopic blood vessel collection system, for example, from a lower limb of a patient.

An endoscopic blood vessel collection system includes a medical device including a blood vessel collection tool. As a medical device, as described in Japanese Patent Application Laid-Open No. 2011-229923, it is possible to consider a configuration having a first jaw and a second jaw that are openable and closable. In this case, the first jaw is provided with a first electrode, and the second jaw is provided with a second electrode. When a blood vessel is collected in this configuration, the first jaw and the second jaw are first brought from an opened state to the closed state inside a body of the patient, and then the first jaw and the second jaw are allowed to pinch the blood vessel. Next, the first electrode and the second electrode are energized, and the pinched blood vessel is cauterized. Next, the blood vessel is cut with a cutter. By moving the blood vessel collection tool to outside the body of the patient, the blood vessel pinched between the first jaw and the second jaw is extracted to outside the body. In this case, a piece of biological tissue collected by the medical device is a blood vessel.

### SUMMARY

To energize the first electrode and the second electrode in the configuration described above, a distal end of a power line is coupled to each of the first electrode and the second electrode. Note herein that, depending on a position of the power line, there is a concern that, when the first jaw and the second jaw change from the closed state to the opened state, the power line is pushed by an edge of one jaw that rotates, among the first jaw and the second jaw, and is pulled in a distal direction. When such a situation occurs, a tensile force is applied to the power line.

It is therefore an object of the present invention to solve the above-described problem.

(1) One aspect of the present invention relates to a medical device including: a first jaw and a second jaw that are openable and closable and pinch a piece of biological tissue when in a closed state, the second jaw being at least rotatable, in which: the first jaw includes: a first support portion; a first proximal portion continuous with a proximal end side of the first support portion; a first main body portion supported by the first support portion; and a first electrode supported by the first support portion, the second jaw includes: a second support portion; a second proximal portion continuous with a proximal end side of the second support portion; a second main body portion supported by the second support portion; and a second electrode supported by the second support portion, the first proximal portion includes a first inner surface and a first outer surface that is a back surface of the first inner surface, the second proximal portion includes a second inner surface that faces the first inner surface when the first jaw and the second jaw are in the closed state and a second outer surface that is a back surface of the second inner surface, a first power line electrically coupled to the first electrode and a second power line electrically coupled to the second electrode are included, and at least a part of the first power line is disposed along the first outer surface. Note that biological tissue in the present invention includes a biological organ that is an aggregate of pieces of biological tissue.

Since at least a part of the first power line runs outside the first proximal portion, when the first jaw and the second jaw change from the closed state to the opened state, it is avoided that the first power line is pushed by an edge portion of the second proximal portion of the second jaw during rotation and pulled in a distal direction. Therefore, application of a tensile force to the first power line is avoided.

(2) In the medical device according to (1) described above, a rotation shaft serving as a rotation center of the second jaw may be further included, and, in the first power line, a part closer to a proximal end side than the rotation shaft may be disposed along the first outer surface.

With this configuration, when the first jaw and the second jaw change from the closed state to the opened state, it is further avoided that the first power line is pulled in the distal direction.

In the medical device according to (1) or (2) described above, the first support portion may include a first support surface that expands in first axis line directions that are longitudinal directions of the first support portion and in first width directions orthogonal to the first axis line directions and supports the first main body portion, and, when the first support portion is divided into a first continuously-provided portion and a first non-continuously-provided portion based on a first straight line that is parallel to the first axis line directions and bisects the first support surface in the first width directions, the first proximal portion may be continuous with a proximal end of the first continuously-provided portion, and the first support portion may include a first cutout formed on the first continuously-provided portion for allowing the first power line to run.

By allowing the first power line to run on the first cutout, it is easy to electrically couple the first power line to the first electrode.

(4) In the medical device according to any one of (1) to (3) described above, a rotation shaft serving as a rotation center of the second jaw and a first tubular body supporting the first jaw and the second jaw via the rotation shaft may be further included, and the first tubular body may include a first extension portion that extends from a distal end of the first tubular body and supports the second jaw via the rotation shaft, and, when the first extension portion is cut in a direction orthogonal to the axis line directions of the first tubular body, the first power line may be in contact with an inner side of an outline of the first tubular body, the outline appearing on a cutting surface of the first extension portion, or may be positioned more inward than the outline.

By setting the first power line at the position described above, it is possible to accommodate the first power line in the first tubular body.

(5) In the medical device according to (4) described above, a second tubular body that is accommodated inside the first tubular body and moves relative to the first tubular body to rotate the second jaw may be further included, and the second tubular body may include a second extension portion that extends from a distal end of the second tubular body and overlaps with the first extension portion inside the first extension portion, and, when the first extension portion and the second extension portion are cut along a direction orthogonal to the axis line directions of the first tubular body and the second tubular body, the first power line may be positioned in a first space formed by the first extension portion, the second extension portion, the outline, and the first proximal portion.

By setting the first power line at the position described above, it is possible to accommodate the first power line in the first space formed between the first tubular body and the second tubular body.

(6) In the medical device according to (5) described above, the second extension portion may include an inner surface facing the first proximal portion, an outer surface facing the first extension portion, and a side surface continuous with the inner surface and the outer surface and extending in the axis line directions of the second extension portion, and, when the second extension portion is viewed in one of the axis line directions of the second tubular body, the side surface may be parallel to an axis line of the rotation shaft.

When the second extension portion is viewed in one of the axis line directions of the second tubular body, the side surface is a surface parallel to the axis line of the rotation shaft. In this case, the first space increases in capacity, compared with a case where the side surface is curved in an arc shape. Therefore, it is easy to accommodate the first power line between the first tubular body and the second tubular body.

(7) In the medical device according to any one of (1) to (6) described above, the first outer surface of the first proximal portion may include a first groove extending in the axis line directions of the first proximal portion for allowing the first power line to be inserted.

By inserting the first power line into the first groove, an exposure amount of the first power line is reduced. Therefore, when the first proximal portion and the second proximal portion are surrounded by a certain member, it is possible to narrow a space for accommodating the first power line. Therefore, it is possible to downsize the medical device.

(8) In the medical device according to any one of (1) to (7) described above, the first proximal portion may include a penetration portion that is opened on the first inner surface and the first outer surface for allowing the first power line to run, and the first power line may be disposed along the first inner surface at a position closer to a distal end than the penetration portion and may be disposed along the first outer surface at a position closer to a proximal end than the penetration portion.

Even with this configuration, when the first jaw and the second jaw change from the closed state to the opened state, it is further avoided that the first power line is pulled toward a distal end side.

(9) In the medical device according to any one of (1) to (8) described above, a rotation shaft serving as a rotation center of the second jaw may be further included, and the first jaw may be rotatable about the rotation shaft serving as the rotation center, and at least a part of the second power line may be disposed along the second outer surface.

Since at least a part of the second power line runs outside the second proximal portion, when the first jaw and the second jaw change from the closed state to the opened state, it is avoided that the second power line is pushed by an edge portion of the first proximal portion and pulled toward a distal portion. Therefore, application of a tensile force to the second power line is avoided.

(10) In the medical device according to (9) described above, when the first jaw and the second jaw are viewed in one of the axis line directions of the medical device, shapes and positions of the second support portion and the second proximal portion may have a rotationally symmetric relationship with shapes and positions of the first support portion and the first proximal portion, and the second power line may be disposed at a rotationally symmetric position with respect to the first power line.

With this configuration, it is possible to acquire, for the second jaw, identical effects to those of the first jaw as described above.

According to the present invention, it is avoided that the first power line is pushed by the edge portion of the second proximal portion of the second jaw during rotation and pulled in the distal direction. Thereby, application of a tensile force to the first power line is avoided.

### BRIEF DESCRIPTION OF DRAWINGS

FIG. 1 is a perspective view of a main part of a medical device.

FIG. 2 is a perspective view of the main part of the medical device, where illustration of a first tubular body is omitted.

FIG. 3 is an exploded perspective view of a collection tool forming the medical device.

FIG. 4 is a cross-sectional perspective view of a main part of a first jaw.

FIG. 5 is a schematic cross-sectional view when a proximal end side of the medical device is cut in a direction orthogonal to axis line directions of the first tubular body, and a distal end side is viewed from the proximal end side.

FIG. 6 is a perspective view of the main part of the first jaw according to a modification example.

FIG. 7 is a schematic side cross-sectional view of the collection tool in a closed state.

FIG. 8 is a schematic side cross-sectional view illustrating a state in which a cutter is advanced in the collection tool in the closed state.

FIG. 9 is a schematic side cross-sectional view of the collection tool in an opened state.

### DESCRIPTION OF EXAMPLE EMBODIMENTS

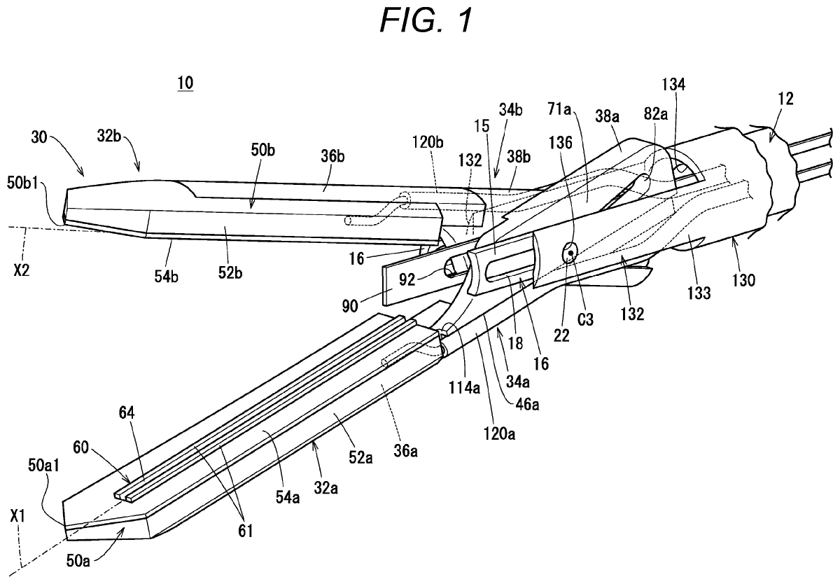

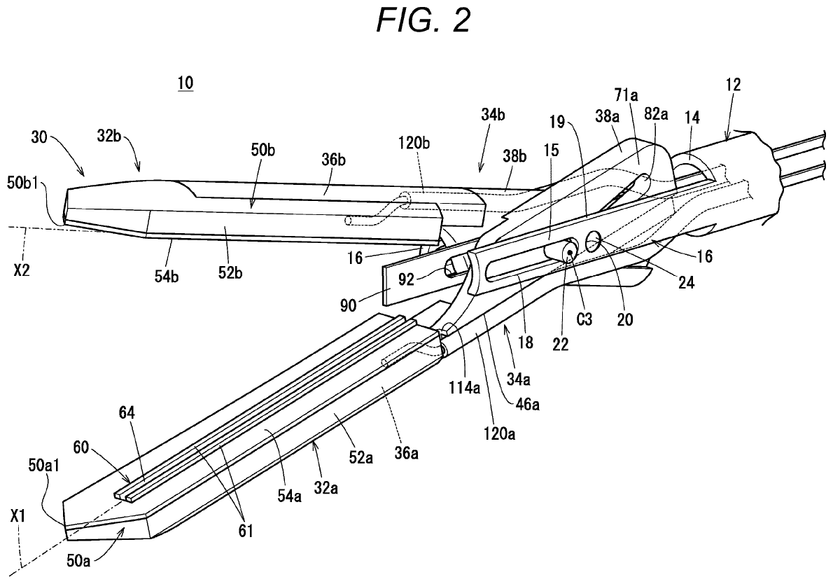

Hereinafter, the term “distal” refers to, in each component, an approaching direction to biological tissue when a piece of the biological tissue is to be collected. The term “proximal” refers to, in each component, an opposite direction to the distal. For example, in a first jaw 32a illustrated in FIGS. 1 and 2, a first pinching portion 50a is positioned relatively closer to a distal end side than a first proximal portion 38a. The first proximal portion 38a is positioned relatively closer to a proximal end side than the first pinching portion 50a.

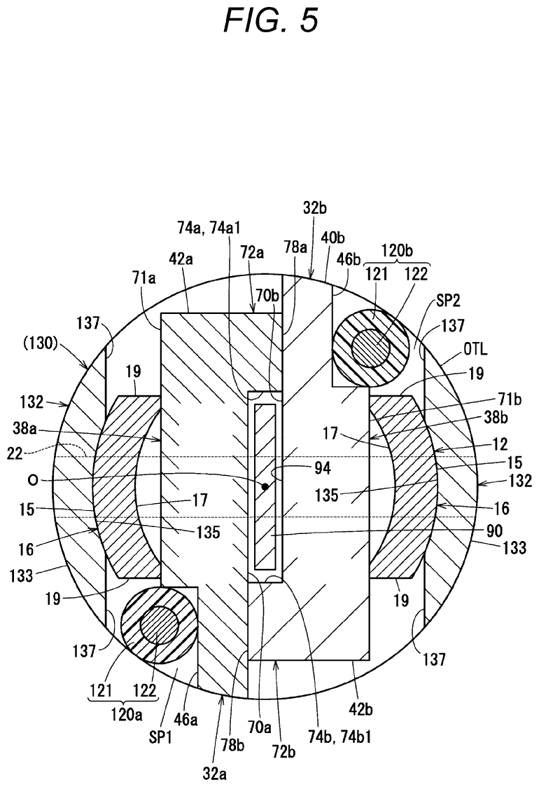

Furthermore, in an embodiment described below, such an aspect is exemplified that both the first jaw 32a and a second jaw 32b are rotatable about a rotation shaft 22 serving as a rotation center. Positions and shapes of the first jaw 32a and the second jaw 32b basically have a rotationally symmetric relationship with respect to each other about a symmetry center O illustrated in FIG. 5. Therefore, for names of components in the second jaw 32b, which are identical to components in the first jaw 32a, the term “first” in the name of each of the components in the first jaw 32a is replaced with “second”. In addition, for the components in the second jaw 32b, which are identical to the components in the first jaw 32a, the alphabet “a” in a reference numeral of each of the components in the first jaw 32a is in principle replaced with “b”. Note that detailed description of the components in the second jaw 32b may be omitted.

The expression “opening and closing of a collection tool 30” is synonymous with “opening and closing of the first jaw 32a and the second jaw 32b”. The expression “the collection tool 30 is in an opened state” is synonymous with “the first jaw 32a and the second jaw 32b are in the opened state”, and “the collection tool 30 is in a closed state” is synonymous with “the first jaw 32a and the second jaw 32b are in the closed state”.

However, the present invention is not limited to the aspects described above. For example, the first jaw 32a may be non-rotatably fixed, and only the second jaw 32b may be rotatable. In this case, for example, first extension portions 132 of a first tubular body 130 and second extension portions 16 of a second tubular body 12 may not necessarily be paired, but may each be a separate component. Note that, also in this case, the collection tool 30 opens and closes as the second jaw 32b rotates.

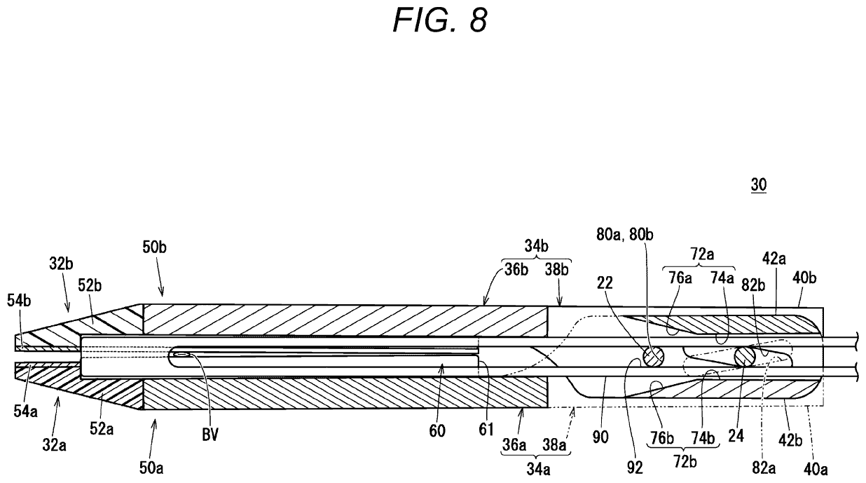

A case where a blood vessel (for example, a branch blood vessel BV illustrated in FIG. 8) is a piece of biological tissue, which is regarded as a target of collection, will now be hereinafter exemplified and described. However, the target of collection is not limited to a blood vessel. The target of collection may be a luminal tube of a living body, other than a blood vessel. Note herein that biological tissue referred to in the present invention includes biological organs. A luminal organ is an example biological organ. Example luminal organs include digestive tract, ureter, trachea, and cervical canal.

FIG. 1 is a perspective view of a main part of a medical device 10 according to the present embodiment. The medical device 10 includes the first tubular body 130, the second tubular body 12, and the collection tool 30. The first tubular body 130 and the second tubular body 12 are tubular members linearly extending in axis line directions. The first tubular body 130 and the second tubular body 12, which each have a cylindrical shape in the illustrated example, may each have a polygonal tubular shape.

The first tubular body 130 includes the pair of first extension portions 132. The pair of first extension portions 132 extend from a distal end of the first tubular body 130 in one of the axis line directions of the first tubular body 130. An outer surface and an inner surface of each of the pair of first extension portions 132 are a curved outer surface 133 and a curved inner surface 135 (see FIG. 5), respectively. A flat side surface 137 is continuous with the curved outer surface 133 and the curved inner surface 135. A pair of first cutout grooves 134 are formed between the pair of first extension portions 132 (see FIG. 1). The pair of first cutout grooves 134 are disposed at positions separated away from each other by an angle of 180° in circumferential directions. The pair of first cutout grooves 134 extend in the axis line directions of the first tubular body 130.

A pair of support holes 136 are formed on the pair of first extension portions 132, respectively. Axis line directions of the support holes 136 are orthogonal to the axis line directions of the first tubular body 130. Each of the support holes 136 penetrates from the curved outer surface 133 to the curved inner surface 135 of each of the first extension portions 132. Center positions of the pair of support holes 136 are shifted by an angle of 90° in the circumferential directions of the first tubular body 130 with respect to centers of the pair of first cutout grooves 134. Both end portions in the axis line directions of the rotation shaft 22 described later are fitted into the pair of support holes 136, respectively. Based on this fitting, the rotation shaft 22 is positioned and fixed with respect to the first tubular body 130.

Although not illustrated, a proximal portion of the first tubular body 130 is provided with, for example, a jaw operation portion, a cutter operation portion, and an energization switch. For example, a first power line 120a and a second power line 120b through which a high-frequency current flows are accommodated in a hollow interior of the first tubular body 130.

The second tubular body 12 is shorter than the first tubular body 130 and is accommodated at the distal end and in the hollow interior of the first tubular body 130. As an operator operates the jaw operation portion, a movement force is transmitted to the second tubular body 12 via a non-illustrated movement force transmission member. As a result, the second tubular body 12 moves inside the first tubular body 130. In this manner, the second tubular body 12 is movably accommodated inside the first tubular body 130.

As illustrated in FIG. 2 in which illustration of the first tubular body 130 is omitted, the second tubular body 12 includes the pair of second extension portions 16. The pair of second extension portions 16 extend from a distal end of the second tubular body 12 in the one of the axis line directions of the second tubular body 12. An outer surface 15 and an inner surface 17 of each of the pair of second extension portions 16 are curved surfaces (see FIG. 5). The pair of second extension portions 16 each include a pair of side surfaces 19 continuous with the outer surface 15 and the inner surface 17 and extending in the axis line directions of the second extension portions 16. The outer surface 15 may be in sliding contact with the curved inner surface 135 of the first tubular body 130.

A pair of second cutout grooves 14 are formed between the pair of second extension portions 16. The pair of second cutout grooves 14 are formed at positions separated away from each other by an angle of 180° in the circumferential directions. The pair of second cutout grooves 14 extend in the axis line directions of the second tubular body 12. The pair of first extension portions 132 cover the pair of second extension portions 16. The pair of second cutout grooves 14 overlap with the pair of first cutout grooves 134.

A pair of guide grooves 18 and a pair of pin attachment holes 20 are formed on the pair of second extension portions 16, respectively. The pair of guide grooves 18 extend in the axis line directions of the second tubular body 12 and each fully penetrate from the outer surface 15 to the inner surface 17 of each of the pair of second extension portions 16. The pair of pin attachment holes 20 are positioned closer to the proximal end side than the pair of guide grooves 18. The pair of pin attachment holes 20 are circular through holes each penetrating from the outer surface 15 to the inner surface 17 of each of the pair of second extension portions 16. Center positions of the pair of guide grooves 18 and the pair of pin attachment holes 20 are shifted by an angle of 90° in the circumferential directions of the second tubular body 12 with respect to centers of the pair of second cutout grooves 14.

The rotation shaft 22 having a cylindrical shape is inserted into each of the pair of guide grooves 18. The rotation shaft 22 moves relative to the second tubular body 12 in the guide grooves 18 along with displacement in one of the axis line directions of the collection tool 30. In other words, the guide grooves 18 allow relative movement of the second tubular body 12 with respect to the rotation shaft 22. Note that, as described above, the end portions of the rotation shaft 22 are fitted into the pair of support holes 136 formed on the pair of first extension portions 132 of the first tubular body 130.

End portions of an open-and-close pin 24 are inserted into the pair of pin attachment holes 20. The open-and-close pin 24 is a pin fixed to the second tubular body 12. When the collection tool 30 is displaced in one of the axis line directions, the open-and-close pin 24 is displaced relative to the first jaw 32a and the second jaw 32b.

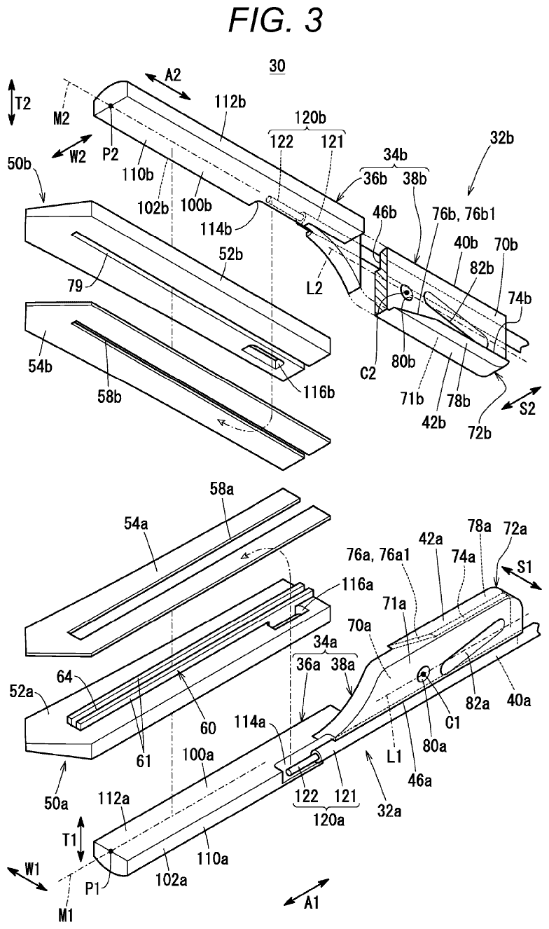

The collection tool 30 includes the first jaw 32a, the second jaw 32b, and a cutter 90. As illustrated in FIG. 3, the first jaw 32a includes a first base member 34a. The first base member 34a integrally includes a first support portion 36a having a flat first support surface 100a and a first proximal portion 38a continuous with the first support portion 36a. Typically, a raw material of the first base member 34a is metal.

The first support portion 36a is positioned on a distal end side of the first base member 34a. The first proximal portion 38a is positioned on a proximal end side of the first base member 34a. Thickness directions T1 of the first support portion 36a are orthogonal to thickness directions S1 of the first proximal portion 38a.

The first support portion 36a includes the first support surface 100a and two side surfaces adjacent to the first support surface 100a in first width directions W1. A first cutout 114a described later is opened on one of the two side surfaces. This side surface will be hereinafter referred to as a first side surface 102a. The first width directions W1 are directions identical to the thickness directions S1 of the first proximal portion 38a.

The first support surface 100a is a flat surface expanding in first axis line directions A1 and the first width directions W1. The first side surface 102a intersects and is substantially orthogonal to an edge portion of the first support surface 100a. The first side surface 102a is a flat surface expanding in the first axis line directions A1 and the thickness directions T1 of the first support portion 36a, which are orthogonal to the first axis line directions A1, which are illustrated in FIG. 3. Note that the first axis line directions A1 are directions extending in longitudinal directions of the first support portion 36a. The thickness directions T1 of the first support portion 36a are directions orthogonal to the first axis line directions A1 and the first width directions W1.

Note herein that FIG. 3 illustrates a first straight line M1 that is parallel to the first axis line directions A1 and bisects the first support surface 100a in the first width directions W1. That is, the first straight line M1 passes through a midpoint P1 in the first width directions W1 of the first support surface 100a. The first support portion 36a is divided into a first continuously-provided portion 110a and a first non-continuously-provided portion 112a based on the first straight line M1. The first continuously-provided portion 110a is a portion where the first proximal portion 38a is continuous on the first support portion 36a, and the first non-continuously-provided portion 112a is a portion where the first proximal portion 38a is not continuous on the first support portion 36a. The first side surface 102a is a side surface on the first support portion 36a, which faces the first continuously-provided portion 110a.

The first support portion 36a includes the first cutout 114a. The first cutout 114a is positioned on the first continuously-provided portion 110a. In the illustrated example, the first cutout 114a is formed by hollowing out a part of the first side surface 102a and a part of the first support surface 100a into a substantially rectangular parallelepiped shape. Therefore, the first cutout 114a is opened on the first support surface 100a and the first side surface 102a. The first cutout 114a is positioned close to a proximal end of the first support portion 36a and close to a distal end of the first proximal portion 38a. However, a shape and a position of the first cutout 114a are not limited to the shape and position described above.

The first support portion 36a forms the first pinching portion 50a. Specifically, the first pinching portion 50a includes the first support portion 36a, a plate-shaped first main body portion 52a, and a first electrode 54a. The first main body portion 52a and the first electrode 54a are supported by the first support portion 36a in a stacked state. The first main body portion 52a includes, for example, an insulator. In this case, the first main body portion 52a is present between the first support portion 36a and the first electrode 54a to accordingly electrically insulate the first support portion 36a and the first electrode 54a from each other.

The first main body portion 52a includes a spacer 60 protruding toward a second support portion 36b. The spacer 60 may be formed integrally with the first main body portion 52a or may be a separate member from the first main body portion 52a. The spacer 60 includes a pair of side wall portions 61. A first cutter groove 64 is formed between the pair of side wall portions 61. The first cutter groove 64 extends in the axis line directions of the first jaw 32a and guides movement of the cutter 90 in one of the axis line directions. A width of the first cutter groove 64 is equal to or slightly larger than a thickness (a size in width directions) of the cutter 90. The pair of side wall portions 61 are present between the cutter 90 inserted into the first cutter groove 64 and the first electrode 54a to electrically insulate the cutter 90 and the first electrode 54a from each other.

A first slit 58a is formed on the first electrode 54a. The pair of side wall portions 61 of the spacer 60 are exposed from the first slit 58a and protrude toward the second support portion 36b.

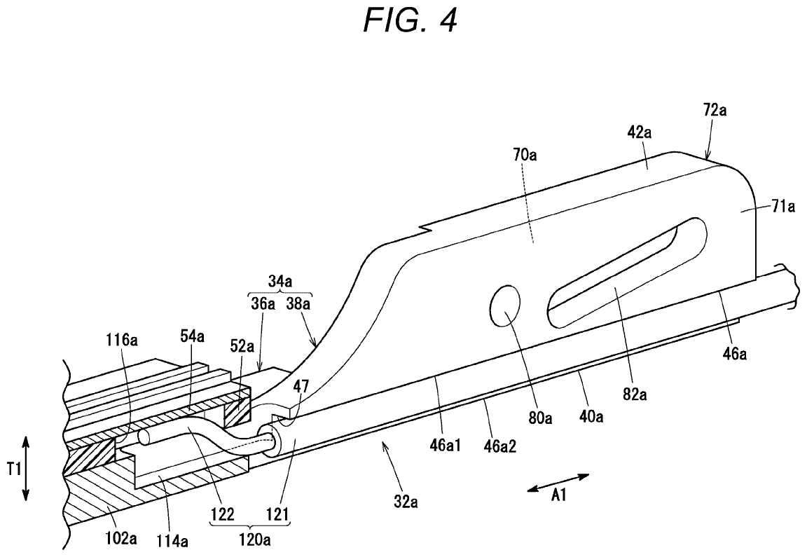

A first through hole 116a penetrating in the thickness directions of the first main body portion 52a is formed on the first main body portion 52a. As illustrated in FIG. 4, a proximal-end-side end portion of the first through hole 116a overlaps with a distal-end-side end portion of the first cutout 114a. Note that the first through hole 116a may wholly overlap with the first cutout 114a wholly. In addition, the first through hole 116a may not overlap with the first cutout 114a.

The first pinching portion 50a includes a first pinching portion axis line X1 illustrated in FIGS. 1 and 2. A most distal portion 50a1 of the first pinching portion 50a may be positioned on the first pinching portion axis line X1 or may be shifted from the first pinching portion axis line X1.

On the other hand, the first proximal portion 38a includes a first extension side end portion 40a (a lower end portion in FIG. 3) to which the first support portion 36a extends and a first non-extension side end portion 42a that is an end portion opposite to the first extension side end portion 40a. Note herein that the first non-extension side end portion 42a is positioned on an opposite side of the first extension side end portion 40a based on a first parallel line L1 passing through a first bearing hole 80a and parallel to an axis line of the first jaw 32a. Note that a center C1 of the first bearing hole 80a is the rotation center of the first jaw 32a. The first support portion 36a extends in the distal direction from a distal end of the first extension side end portion 40a of the first proximal portion 38a and is not positioned on the first non-extension side end portion 42a.

The first proximal portion 38a has a flange shape wider than the first support portion 36a. The first proximal portion 38a includes a flat first inner surface 70a and a flat first outer surface 71a. The first inner surface 70a and the first outer surface 71a have a front-back relationship in the thickness directions S1 of the first proximal portion 38a.

The first inner surface 70a expands in the axis line directions of the first proximal portion 38a and directions orthogonal to the first width directions W1 of the first support surface 100a. The first inner surface 70a includes a first protrusion portion 72a protruding in a direction orthogonal to an axis line of the first proximal portion 38a (or the axis line of the first jaw 32a). The first protrusion portion 72a is provided on the first non-extension side end portion 42a of the first proximal portion 38a. The first protrusion portion 72a includes a first linear-shaped portion 74a and a first inclined portion 76a. The first linear-shaped portion 74a extends linearly along the axis line from a proximal end toward the distal end of the first proximal portion 38a. The first linear-shaped portion 74a includes a flat surface 74a1 (see FIG. 5) having a width parallel to the center C1 of the first bearing hole 80a. The flat surface 74a1 extends parallel to the symmetry center O. The width of the flat surface 74a1 corresponds to a protrusion amount of the first protrusion portion 72a. The first inclined portion 76a is inclined in a direction of separation from the first parallel line L1 as heading toward the distal end of the first proximal portion 38a. The first inclined portion 76a is formed to include a flat surface 76a1 (see FIG. 3) having a width parallel to the center C1 of the first bearing hole 80a. The width of the flat surface 76a1 corresponds to the protrusion amount of the first protrusion portion 72a.

The first protrusion portion 72a includes a first sliding contact surface 78a. As will be understood from FIG. 5, when the collection tool 30 is in the closed state, the first sliding contact surface 78a faces a second inner surface 70b of a second proximal portion 38b forming the second jaw 32b. As illustrated in FIGS. 3 and 4, the first bearing hole 80a and a first sliding groove 82a are formed on the first proximal portion 38a.

The first outer surface 71a includes a first groove 46a. The first groove 46a extends in the axis line directions of the first proximal portion 38a on the first extension side end portion 40a of the first outer surface 71a. As illustrated in FIG. 4, the first groove 46a includes a side portion 46a1 that is one end in directions orthogonal to the axis line directions of the first proximal portion 38a and orthogonal to the thickness directions S1 of the first proximal portion 38a and a side portion 46a2 that is another end in the directions orthogonal to the axis line directions of the first proximal portion 38a and orthogonal to the thickness directions S1 of the first proximal portion 38a. The side portion 46a1 is closer to the first non-extension side end portion 42a than the side portion 46a2. The side portion 46a1 includes a step 47 formed based on a recess from an outer surface of the first outer surface 71a to a bottom surface of the first groove 46a. On the other hand, the side portion 46a2 forms an opened space that is opened at an edge portion in the axis line directions of the first extension side end portion 40a and has no step. That is, the side portion 46a2 is an open end. In this manner, since the side portion 46a2 is the open end that forms the opened space, even when the first proximal portion 38a moves upward in FIG. 5 when the first jaw 32a changes from the closed state to the opened state, the first proximal portion 38a is prevented from being caught by the first power line 120a. Thereby, application of a tensile force to the first power line 120a is avoided.

The first power line 120a is electrically coupled to the first electrode 54a. Specifically, a conductive wire 122 is exposed from an insulating coating 121 at a distal end of the first power line 120a. The conductive wire 122 is at least allowed to pass through the first cutout 114a formed on the first support portion 36a, and is then allowed to be inserted into the first through hole 116a formed on the first main body portion 52a. In the first through hole 116a, a part of a surface of the first electrode 54a, which faces the first main body portion 52a, is exposed. The conductive wire 122 is bonded to a portion of the first electrode 54a, which is exposed in the first through hole 116a.

As illustrated in FIG. 4, the insulating coating 121 exposed from the first cutout 114a at the distal end of the first power line 120a is inserted into the first groove 46a described above. With this insertion, the first power line 120a is disposed along the first outer surface 71a of the first proximal portion 38a. Note that it is not necessary to always form the first groove 46a on the first proximal portion 38a. A side surface of the insulating coating 121 may abut the flat first outer surface 71a on which the first groove 46a is not formed.

The first groove 46a linearly extends from a most distal end to a most proximal end of the first proximal portion 38a on the first extension side end portion 40a of the first proximal portion 38a. Therefore, in this case, at the distal end of the first power line 120a, both a portion closer to the distal end side than the rotation shaft 22 serving as the rotation center of the first jaw 32a and a portion closer to the proximal end side than the rotation shaft 22 are disposed along the first outer surface 71a.

Instead of forming the first cutout 114a on the first support portion 36a, an insertion port continuous with the first through hole 116a may be formed at a proximal end of the first main body portion 52a. In this case, the first power line 120a is inserted from the insertion port, and the conductive wire 122 is bonded to the portion of the first electrode 54a, which is exposed in the first through hole 116a.

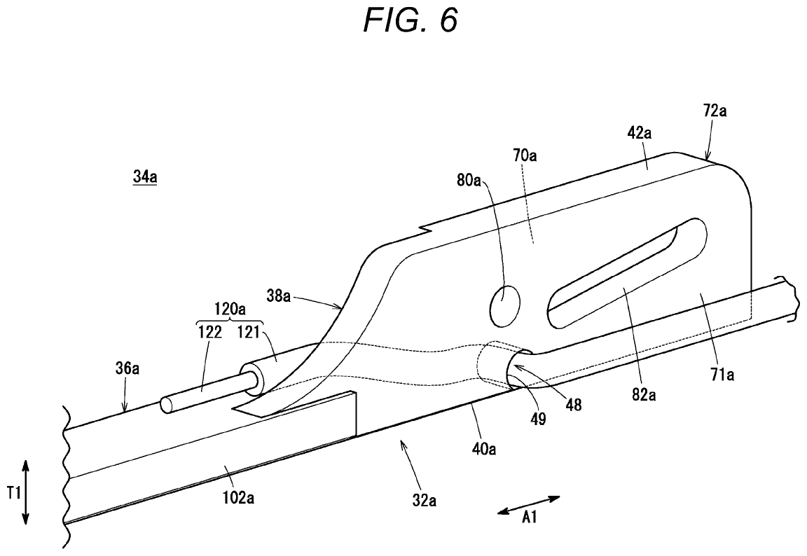

As illustrated in FIG. 6, a penetration portion 48 may be formed on the first proximal portion 38a. The penetration portion 48 is formed, for example, at a position closer to the first extension side end portion 40a than the first bearing hole 80a through which the rotation shaft 22 passes. In the illustrated example, the penetration portion 48 is a through hole 49. The through hole 49 may be positioned closer to the distal end side than the first bearing hole 80a or closer to the proximal end side than the first bearing hole 80a in the axis line directions of the first proximal portion 38a. Alternatively, the through hole 49 may be parallel to the first bearing hole 80a in the axis line directions of the first proximal portion 38a and at a position closer to the first extension side end portion 40a than the first bearing hole 80a.

In this case, the first power line 120a is allowed to pass through the through hole 49. The first power line 120a is disposed along the first inner surface 70a of the first proximal portion 38a for a portion closer to the distal end than the through hole 49 and is disposed along the first outer surface 71a of the first proximal portion 38a for a portion closer to the proximal end than the through hole 49. Note that both the first cutout 114a and the penetration portion 48 may be formed on the first jaw 32a.

The penetration portion 48 may be a cutout having a shape acquired by cutting out a part of the first extension side end portion 40a of the first proximal portion 38a. To allow the distal end of the first power line 120a to pass through the penetration portion 48 to couple the conductive wire 122 to the first electrode 54a, an insertion port continuous with the first through hole 116a is formed at the proximal end of the first main body portion 52a, similar to the those described above, for example. In this case, the first power line 120a is inserted from the insertion port, and the conductive wire 122 is bonded to the portion of the first electrode 54a, which is exposed in the first through hole 116a.

A surface of the first support portion 36a may be covered with an insulator. Thereby, conduction between the conductive wire 122 and the first support portion 36a is avoided. Alternatively, the conductive wire 122 coupled to the first electrode 54a may be covered with an insulator such as silicone resin. In the case of the aspect illustrated in FIG. 4, the first cutout 114a may be filled with a silicone resin for this purpose, for example.

A second base member 34b of the second jaw 32b integrally includes the second support portion 36b and the second proximal portion 38b. The second support portion 36b forms a second pinching portion 50b together with a second main body portion 52b and a second electrode 54b.

As illustrated in FIG. 3, the second support portion 36b includes a second support surface 100b and a second side surface 102b adjacent to the second support surface 100b in second width directions W2. The second side surface 102b is a flat surface expanding in second axis line directions A2 and thickness directions T2 of the second support portion 36b, which are orthogonal to second axis line directions A2. Note that the second axis line directions A2 are directions extending in longitudinal directions of the second support portion 36b. The thickness directions T2 of the second support portion 36b are directions orthogonal to the second axis line directions A2 and the second width directions W2. The second width directions W2 are directions identical to the thickness directions S2 of the second proximal portion 38b.

FIG. 3 illustrates a second straight line M2 that is parallel to the second axis line directions A2 and bisects the second support surface 100b in the second width directions W2. That is, the second straight line M2 passes through a midpoint P2 in the second width directions W2 of the second support surface 100b. The second support portion 36b is divided into a second continuously-provided portion 110b and a second non-continuously-provided portion 112b based on the second straight line M2. The second proximal portion 38b is continuous with a proximal end of the second continuously-provided portion 110b and is not positioned on the second non-continuously-provided portion 112b. The second side surface 102b faces the second continuously-provided portion 110b.

The second support portion 36b includes a second cutout 114b. The second cutout 114b is positioned on the second continuously-provided portion 110b. In the illustrated example, the second cutout 114b is formed by hollowing out a part of the second side surface 102b and a part of the second support surface 100b into a substantially rectangular parallelepiped shape. The second cutout 114b is positioned near a proximal end of the second support portion 36b and near a distal end of the second proximal portion 38b. However, a shape and a position of the second cutout 114b are not limited to the shape and position described above.

As illustrated in FIG. 3, a second cutter groove 79 and a second slit 58b are formed on the second main body portion 52b and the second electrode 54b, respectively. The second slit 58b overlaps with the second cutter groove 79. The second slit 58b is wider than the first cutter groove 64. Therefore, when the cutter 90 moves into the first cutter groove 64, side surfaces of the cutter 90 are prevented from coming into contact with inner surfaces of the second slit 58b. In other words, the side surfaces of the cutter 90 and side surfaces of the second slit 58b do not come in contact with each other. Based on this non-contact feature, the cutter 90 and the second electrode 54b are electrically insulated from each other.

A second through hole 116b penetrating in the thickness directions of the second main body portion 52b is formed on the second main body portion 52b. Similar to those illustrated in FIG. 4, a proximal-end-side end portion of the second through hole 116b overlaps with a distal-end-side end portion of the second cutout 114b. Note that the second through hole 116b may wholly overlap with the second cutout 114b wholly. In addition, the second through hole 116b may not overlap with the second cutout 114b.

The second pinching portion 50b includes a second pinching portion axis line X2 illustrated in FIGS. 1 and 2. A most distal portion 50b1 of the second pinching portion 50b may be positioned on the second pinching portion axis line X2 or may be shifted from the second pinching portion axis line X2. In the latter case, it is preferable that a direction of deviation of the most distal portion 50a1 of the first pinching portion 50a with respect to the first pinching portion axis line X1 and a direction of deviation of the most distal portion 50b1 of the second pinching portion 50b with respect to the second pinching portion axis line X2 are identical directions.

As illustrated in FIG. 3, a second protrusion portion 72b provided on the second non-extension side end portion 42b of the second proximal portion 38b includes a second linear-shaped portion 74b, a second inclined portion 76b, and a second sliding contact surface 78b. The second linear-shaped portion 74b includes a flat surface 74b1 (see FIG. 5) having a width parallel to a center C2 of a second bearing hole 80b. The flat surface 74b1 extends parallel to the symmetry center O. The width of the flat surface 74b1 corresponds to a protrusion amount of the second protrusion portion 72b. The second inclined portion 76b is inclined in a direction of separation from a second parallel line L2 as heading toward the distal end of the second proximal portion 38b. The second inclined portion 76b is formed to include a flat surface 76b1 (see FIG. 3) having a width parallel to the center C2 of the second bearing hole 80b. The width of the flat surface 76b1 corresponds to the protrusion amount of the second protrusion portion 72b.

As will be understood from FIG. 5, when the collection tool 30 is in the closed state, the second sliding contact surface 78b faces the first inner surface 70a of the first proximal portion 38a. When the first jaw 32a and the second jaw 32b close, the first sliding contact surface 78a of the first protrusion portion 72a is in sliding contact with the second inner surface 70b on the second extension side end portion 40b of the second proximal portion 38b. Note herein that the second non-extension side end portion 42b is positioned on an opposite side of the second extension side end portion 40b based on the second parallel line L2 (see FIG. 3) passing through the second bearing hole 80b and parallel to an axis line of the second jaw 32b. The second sliding contact surface 78b of the second protrusion portion 72b is in sliding contact with the first inner surface 70a on the first extension side end portion 40a of the first proximal portion 38a.

The conductive wire 122 forming a distal end of the second power line 120b passes through the second cutout 114b (see FIG. 3). The conductive wire 122 of the second power line 120b is drawn out to the second through hole 116b via the second cutout 114b. In the second through hole 116b, a part of a surface of the second electrode 54b, which faces the second main body portion 52b, is exposed. The conductive wire 122 is bonded to a portion of the second electrode 54b, which is exposed in the second through hole 116b.

A second groove 46b is formed on a second outer surface 71b of the second proximal portion 38b. The insulating coating 121 exposed from the second cutout 114b at the distal end of the second power line 120b is inserted into the second groove 46b. With this insertion, the second power line 120b is disposed along the second outer surface 71b of the second proximal portion 38b. This disposition is a mere example, similar to those described above, and it is not necessary to always form the second groove 46b on the second proximal portion 38b. For example, a side surface of the insulating coating 121 of the second power line 120b may abut the flat second outer surface 71b where the second groove 46b is not formed.

Instead of the second cutout 114b, an insertion port continuous with the second through hole 116b may be formed at a proximal end of the second main body portion 52b. Alternatively, similar to FIG. 6, the penetration portion 48 may be formed on the second proximal portion 38b.

In the second jaw 32b, portions for which detailed descriptions are omitted are configured similarly to the first jaw 32a.

The first inner surface 70a and the second inner surface 70b are separated away from each other by a distance corresponding to each of the protrusion amounts of the first protrusion portion 72a and the second protrusion portion 72b (see FIG. 5). Therefore, a cutter space 94 is formed by the first inner surface 70a, the first protrusion portion 72a, the second inner surface 70b, and the second protrusion portion 72b. Specifically, the cutter space 94 is formed by the first inner surface 70a, the flat surface 74b1 of the second linear-shaped portion 74b, the second inner surface 70b, and the flat surface 74a1 of the first linear-shaped portion 74a.

Note herein that FIG. 5 is a schematic cross-sectional view when a proximal end side of the medical device 10 is cut in a direction orthogonal to the axis line directions of the first tubular body 130 (the width directions or radial directions), and a distal end side is viewed from the proximal end side. An outline OTL of the first tubular body 130 appears on cutting surfaces of the pair of first extension portions 132. The outline OTL is in contact with the curved outer surfaces 133 of the pair of first extension portions 132. A space surrounded by the flat side surface 137 of one of the pair of first extension portions 132, the side surface 19 of one of the pair of second extension portions 16, the outline OTL, and the first proximal portion 38a is a first space SP1. A space surrounded by the flat side surface 137 of the other of the pair of first extension portions 132, the side surface 19 of the other of the pair of second extension portions 16, the outline OTL, and the second proximal portion 38b is a second space SP2.

The first power line 120a is positioned in the first space SP1, and the second power line 120b is positioned in the second space SP2. Therefore, the first power line 120a and the second power line 120b are positioned more inward than the outline OTL. Alternatively, a side surface of the first power line 120a or a side surface of the second power line 120b may be in contact with an inner side of the outline OTL.

As illustrated in FIG. 3, the second bearing hole 80b and a second sliding groove 82b are formed on the second proximal portion 38b. As illustrated in FIG. 2, when the first proximal portion 38a of the first jaw 32a and the second proximal portion 38b of the second jaw 32b are inserted into the second cutout grooves 14 of the second tubular body 12 and pinched between the pair of second extension portions 16, the first bearing hole 80a and the second bearing hole 80b are pinched between the pair of guide grooves 18. Therefore, the rotation shaft 22 is allowed to pass through one of the pair of support holes 136, one of the pair of guide grooves 18, the first bearing hole 80a, an elongated hole 92 (described later) formed on the cutter 90, the second bearing hole 80b, the other of the pair of guide grooves 18, and the other of the pair of support holes 136. The rotation shaft 22 couples the first proximal portion 38a of the first jaw 32a and the second proximal portion 38b of the second jaw 32b to each other.

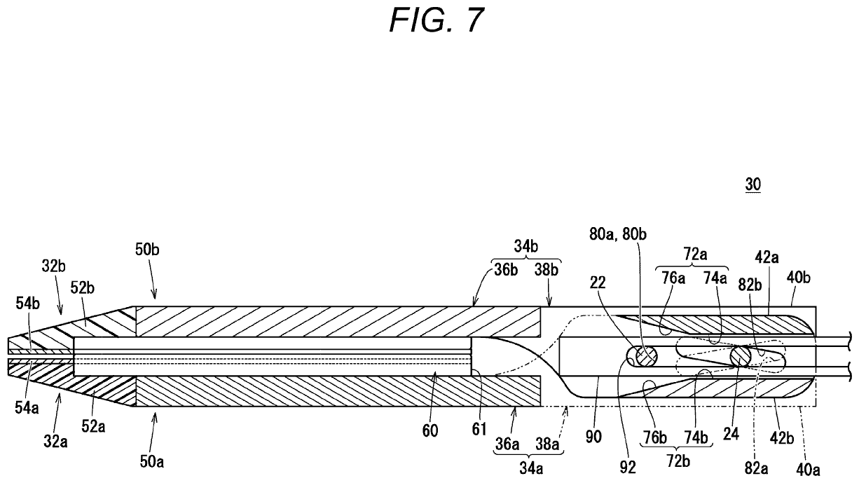

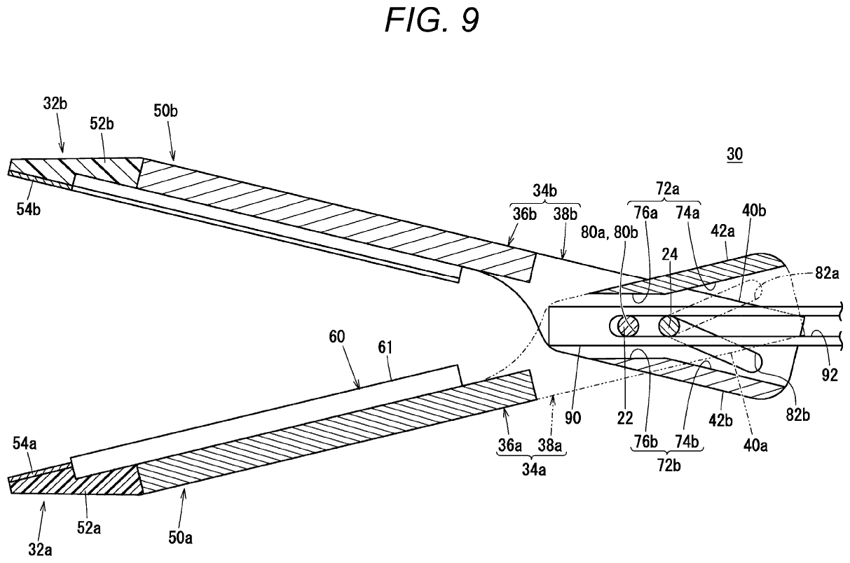

As illustrated in FIG. 3, the first sliding groove 82a is positioned, on the first proximal portion 38a, closer to the proximal end side than the first bearing hole 80a. The first sliding groove 82a is inclined from the first extension side end portion 40a toward the first non-extension side end portion 42a as heading from the distal end toward the proximal end. The second sliding groove 82b formed on the second proximal portion 38b is similar to those described above. However, since the first jaw 32a and the second jaw 32b have a rotationally symmetric relationship, when the first jaw 32a and the second jaw 32b are in the opened state, the first sliding groove 82a and the second sliding groove 82b form a V shape with its opening facing the proximal end (see FIG. 9). On the other hand, as illustrated in FIG. 7, when the first jaw 32a and the second jaw 32b are in the closed state, the first sliding groove 82a and the second sliding groove 82b intersect each other to form an X shape.

As illustrated in FIG. 2, when the first proximal portion 38a of the first jaw 32a and the second proximal portion 38b of the second jaw 32b are pinched between the pair of second extension portions 16 of the second tubular body 12, the pair of pin attachment holes 20 overlap with predetermined positions of the first sliding groove 82a and the second sliding groove 82b. Therefore, the open-and-close pin 24 is allowed to pass through one of the pair of pin attachment holes 20, the first sliding groove 82a, the elongated hole 92, the second sliding groove 82b, and the other of the pair of pin attachment holes 20. As will be described later, as the open-and-close pin 24 moves relative to the first sliding groove 82a and the second sliding groove 82b, the first jaw 32a and the second jaw 32b are opened or closed.

The cutter 90 is disposed between the first proximal portion 38a and the second proximal portion 38b. The cutter 90 extends in the axis line directions of the first tubular body 130 and the second tubular body 12. The cutter 90 is positioned close to the proximal end side in an initial state. Note herein that the cutter operation portion is provided on the proximal portion (not illustrated) of the first tubular body 130, as described above. When the operator operates the cutter operation portion, it is possible to move the cutter 90 forward or backward in the distal direction or a proximal direction of the axis line directions. Note that, when the collection tool 30 is to be in the closed state, the cutter 90 moves toward the distal end in the one of the axis line directions along the first cutter groove 64 and the second cutter groove 79 in the cutter space 94 illustrated in FIG. 5.

As illustrated in FIG. 1, the cutter 90 has the elongated hole 92 penetrating in the thickness directions of the cutter 90. The rotation shaft 22 and the open-and-close pin 24 are allowed to pass through the elongated hole 92.

The medical device 10 according to the present embodiment is basically configured as described above. Next, collection of a blood vessel using the medical device 10 will now be described herein. Note that the operator in the below description is, for example, a medical operator, and is typically a doctor.

To collect a blood vessel such as a branch blood vessel BV (see FIG. 8) from a body of a patient, the medical device 10 and an imaging device are inserted into the body of the patient. The medical device 10 is brought to a position close to the branch blood vessel BV that is a target of collection, and the medical device 10 is brought into the opened state illustrated in FIG. 9. In other words, the first pinching portion 50a of the first jaw 32a and the second pinching portion 50b of the second jaw 32b are separated away from each other. At this point in time, the collection tool 30 is positioned close to the proximal end side of the second cutout grooves 14 of the second tubular body 12. The operator disposes the collection tool 30 in the opened state at a position close to the predetermined branch blood vessel BV while performing observation with the imaging device.

While the first jaw 32a and the second jaw 32b are in the opened state, the first inclined portion 76a and the second inclined portion 76b serve as escape portions where no interference with a distal end of the cutter 90 occurs, as illustrated in FIG. 9. In other words, even when the first jaw 32a and the second jaw 32b are in the opened state when the cutter 90 is at a most retracted position, interference of the first protrusion portion 72a and the second protrusion portion 72b with the cutter 90 is avoided.

Next, the operator operates the jaw operation portion described above. With this operation, the second tubular body 12 moves toward the proximal end side inside the first tubular body 130. Therefore, the first jaw 32a and the second jaw 32b are relatively displaced toward the distal end side in the second cutout groove 14.

Note herein that the open-and-close pin 24 is immovable with respect to the second tubular body 12. Therefore, the open-and-close pin 24 moves relative to the first sliding groove 82a of the first jaw 32a and the second sliding groove 82b of the second jaw 32b toward the proximal end side. With this relative movement, the first jaw 32a and the second jaw 32b rotate about a center C3 of the rotation shaft 22, which serves as the rotation center.

In this case, rotation directions of the first jaw 32a and the second jaw 32b are directions in which the first pinching portion 50a and the second pinching portion 50b approach each other. That is, the first jaw 32a and the second jaw 32b rotate in directions for closing. With this rotation, the first sliding contact surface 78a of the first protrusion portion 72a on the first proximal portion 38a comes into sliding contact with the second inner surface 70b of the second proximal portion 38b, and the second sliding contact surface 78b of the second protrusion portion 72b on the second proximal portion 38b comes into sliding contact with the first inner surface 70a of the first proximal portion 38a.

With the rotation described above, the first jaw 32a and the second jaw 32b change from the opened state to the closed state, as illustrated in FIGS. 7 and 8. Along with this change, the branch blood vessel BV (see FIG. 8) is pinched between the first pinching portion 50a and the second pinching portion 50b. On the other hand, the pair of side wall portions 61 of the spacer 60, which are exposed from the first slit 58a, abut the second electrode 54b. With this abutment, the first electrode 54a and the second electrode 54b are slightly separated away from each other. In addition, the first cutter groove 64 and the second cutter groove 79 face each other.

Next, the operator operates the energization switch provided on the proximal end portion of the second tubular body 12. Thereby, a high-frequency current is supplied to the first electrode 54a and the second electrode 54b. As a result, the branch blood vessel BV pinched between the first pinching portion 50a and the second pinching portion 50b is cauterized for hemostasis.

Next, the operator operates the cutter operation portion described above. At this time, the collection tool 30 is held in the closed state. Therefore, the cutter 90 advances from the position on the proximal end side illustrated in FIG. 7 toward the distal end in the cutter space 94 illustrated in FIG. 5, and is inserted into the first cutter groove 64 and the second cutter groove 79. The cutter 90 further advances along the first cutter groove 64 and the second cutter groove 79 as illustrated in FIG. 8, and cuts the branch blood vessel BV pinched between the first pinching portion 50a and the second pinching portion 50b. If necessary, the medical device 10 may be further advanced in the body of the patient to cut another branch blood vessel or exfoliate surrounding tissue.

It is possible to move the cutter 90 even when the collection tool 30 is in the opened state. As illustrated in FIG. 9, when the collection tool 30 is in the opened state, the first inclined portion 76a and the second inclined portion 76b are inclined in directions of avoiding interference with the cutter 90. Therefore, when the cutter 90 advances in this state, it is possible to allow the cutter 90 to pass through between the first inclined portion 76a and the second inclined portion 76b. That is, based on the provision of the first inclined portion 76a and the second inclined portion 76b, interference of the first protrusion portion 72a or the second protrusion portion 72b with the cutter 90 is avoided.

After cutting of the branch blood vessel within a desired range has been completed, the medical device 10 and the imaging device are withdrawn from the body of the patient. As described above, main operation of collecting a blood vessel is completed.

The present embodiment achieves those effects described below.

The medical device 10 includes the first jaw 32a and the second jaw 32b that are openable and closable and pinch a piece of biological tissue when in the closed state. At least the second jaw 32b among both the jaws 32a and 32b is rotatable.

The first jaw 32a includes the first support portion 36a, the first proximal portion 38a continuous with the proximal end side of the first support portion 36a, the first main body portion 52a supported by the first support portion 36a, and the first electrode 54a supported by the first support portion 36a. The second jaw 32b includes the second support portion 36b, the second proximal portion 38b continuous with the proximal end side of the second support portion 36b, the second main body portion 52b supported by the second support portion 36b, and the second electrode 54b supported by the second support portion 36b.

The first proximal portion 38a includes the first inner surface 70a and the first outer surface 71a that is the back surface of the first inner surface 70a. The second proximal portion 38b includes the second inner surface 70b and the second outer surface 71b that is the back surface of the second inner surface 70b. The first inner surface 70a and the second inner surface 70b face each other when the first jaw 32a and the second jaw 32b are in the closed state.

The medical device 10 includes the first power line 120a electrically coupled to the first electrode 54a and the second power line 120b electrically coupled to the second electrode 54b. In the configuration described above, at least a part of the first power line 120a is disposed along the first outer surface 71a.

Since at least a part of the first power line 120a runs outside the first proximal portion 38a, when the first jaw 32a and the second jaw 32b change from the closed state to the opened state, it is avoided that the first power line 120a is pushed by the edge portion of the second proximal portion 38b of the second jaw 32b during rotation and pulled in the distal direction. Therefore, application of a tensile force to the first power line 120a is avoided.

The medical device 10 includes the rotation shaft 22 serving as the rotation center of the second jaw 32b. In the first power line 120a, a portion closer to the proximal end side than the rotation shaft 22 is disposed along the first outer surface 71a.

With this configuration, when the first jaw 32a and the second jaw 32b change from the closed state to the opened state, it is further avoided that the first power line 120a is pulled in the distal direction.

The first support portion 36a includes the first support surface 100a that expands in the first axis line directions A1 that are the longitudinal directions of the first support portion 36a and the first width directions W1 orthogonal to the first axis line directions A1 and supports the first main body portion 52a. When the first support portion 36a is divided into the first continuously-provided portion 110a and the first non-continuously-provided portion 112a based on the first straight line M1 that is parallel to the first axis line directions A1 and bisects the first support surface 100a in the first width directions W1, the first proximal portion 38a is continuous with the proximal end of the first continuously-provided portion 110a. The first support portion 36a includes the first cutout 114a formed on the first continuously-provided portion 110a for allowing the first power line 120a to run.

By allowing the first power line 120a to pass through the first cutout 114a, it is easy to electrically couple the first power line 120a to the first electrode 54a.

The medical device 10 includes the rotation shaft 22 serving as the rotation center of the second jaw 32b and the first tubular body 130 supporting the first jaw 32a and the second jaw 32b via the rotation shaft 22. The first tubular body 130 includes the first extension portions 132 that extend from the distal end of the first tubular body 130 and support the second jaw 32b via the rotation shaft 22. When the first extension portions 132 are cut in the direction orthogonal to the axis line directions of the first tubular body 130, the first power line 120a is in contact with the inner side of the outline OTL of the first tubular body 130, which appears on the cutting surfaces of the first extension portions 132. Alternatively, the first power line 120a is positioned more inward than the outline OTL.

By disposing the first power line 120a at the position described above, it is possible to accommodate the first power line 120a in the first tubular body 130.

The medical device 10 includes the second tubular body 12 that is accommodated in the first tubular body 130 and moves relative to the first tubular body 130 to rotate the second jaw 32b. The second tubular body 12 includes the second extension portions 16 that extend from the distal end of the second tubular body 12 and overlap with the first extension portions 132 inside the first extension portions 132. When the first extension portions 132 and the second extension portions 16 are cut in the direction orthogonal to the axis line directions of the first tubular body 130 and the second tubular body 12, the first power line 120a is positioned in the first space SP1 formed by one of the first extension portions 132, one of the second extension portions 16, the outline OTL, and the first proximal portion 38a.

By disposing the first power line 120a at the position described above, it is possible to accommodate the first power line 120a in the first space SP1 formed between the first tubular body 130 and the second tubular body 12.

The second extension portions 16 each include the inner surface 17 facing the first proximal portion 38a, the outer surface 15 facing corresponding one of the first extension portions 132, and the side surface 19 that is continuous with the inner surface 17 and the outer surface 15 and extends in the axis line directions of the second extension portions 16. When the second extension portions 16 are viewed in one of the axis line directions of the second tubular body 12, the side surface 19 is parallel to the axis line of the rotation shaft 22.

When the second extension portions 16 are viewed in one of the axis line directions of the second tubular body 12, the side surface 19 is a surface parallel to the axis line of the rotation shaft 22. In this case, the first space SP1 increases in capacity, compared with a case where the side surface 19 is curved in an arc shape. Therefore, it is easy to accommodate the first power line 120a between the first tubular body 130 and the second tubular body 12.

The first outer surface 71a of the first proximal portion 38a includes the first groove 46a that extends in the axis line directions of the first proximal portion 38a, into which the first power line 120a is inserted.

By inserting the first power line 120a into the first groove 46a, an exposure amount of the first power line 120a is reduced. Therefore, when the first proximal portion 38a and the second proximal portion 38b are surrounded by a certain member, it is possible to narrow a space for accommodating the first power line 120a. Therefore, it is possible to downsize the medical device 10.

The first proximal portion 38a includes the penetration portion 48 that is opened on the first inner surface 70a and the first outer surface 71a, through which the first power line 120a passes (see FIG. 6). The first power line 120a is disposed along the first inner surface 70a at a position closer to the distal end than the penetration portion 48 and is disposed along the first outer surface 71a at a position closer to the proximal end than the penetration portion 48.

Even with this configuration, when the first jaw 32a and the second jaw 32b change from the closed state to the opened state, it is further avoided that the first power line 120a is pulled toward the distal end side.

The medical device 10 includes the rotation shaft 22 serving as the rotation center of the second jaw 32b. The first jaw 32a is rotatable about the rotation shaft 22 serving as the rotation center, and at least a part of the second power line 120b is disposed along the second outer surface 71b.

Since at least a part of the second power line 120b runs outside the second proximal portion 38b, when the first jaw 32a and the second jaw 32b change from the closed state to the opened state, it is avoided that the second power line 120b is pushed by the edge portion of the first proximal portion 38a and pulled toward the distal portion. Therefore, application of a tensile force to the second power line 120b is avoided.

When the first jaw 32a and the second jaw 32b are viewed from one of the axis line directions of the medical device 10, the shapes and positions of the second support portion 36b and the second proximal portion 38b have a rotationally symmetric relationship with the shapes and positions of the first support portion 36a and the first proximal portion 38a. The second power line 120b is disposed at a position rotationally symmetric with the first power line 120a.

With this configuration, it is possible to acquire, for the second jaw 32b, identical effects to those of the first jaw 32a as described above.

Note that, the present invention is not limited to the above disclosure, and various configurations can be adopted without departing from the gist of the present invention. For example, although, in the embodiment described above, a blood vessel that is an example of a luminal organ is exemplified as a piece of biological tissue, as described above, a target of collection may be a piece of biological tissue other than a blood vessel. A piece of biological tissue includes a biological organ. Example biological organs include solid organs or luminal organs. Example luminal organs other than a blood vessel include digestive tract, ureter, trachea, and cervical canal.

1. A medical device comprising:
a first jaw comprising:
a first outer side surface;
a first inner surface opposite of the first outer side surface; and
a first electrode;
a second jaw comprising:
a second outer side surface;
a second inner surface opposite of the second outer side surface and facing the first inner surface; and
a second electrode,
wherein the first and second jaws are reconfigurable between: (i) a closed state in which the first electrode is adjacent to the second electrode to pinch tissue therebetween and (ii) an opened state in which the first and second electrodes are separated farther from each other than in the closed state;
a first power line electrically coupled to the first electrode; and
a second power line electrically coupled to the second electrode,
wherein a portion of the first power line extends along the first outer side surface.

2. The medical device of claim 1, wherein the portion of the first power line is disposed in a first groove defined along the first outer side surface.

3. The medical device of claim 2, wherein the first power line exits a distal end of the first groove and then passes into a first hole that is defined by the first jaw, and wherein the first power line is electrically coupled to the first electrode in the first hole.

4. The medical device of claim 1, further comprising a rotation shaft about which at least one of the first jaw and the second jaw rotate during reconfiguration between the open and closed states, wherein at least some of the portion of the first power line extending along the first outer side surface is proximal of the rotation shaft.

5. The medical device of claim 4, further comprising a first tubular body having a first pair of distally-extending extension portions to which the rotation shaft is mounted, wherein at least some of the portion of the first power line extending along the first outer side surface is disposed between the first outer side surface and a first extension portion of the first pair of distally-extending extension portions.

6. The medical device of claim 5, further comprising a second tubular body slidably disposed within the first tubular body and having a second pair of distally-extending extension portions, wherein the rotation shaft extends through slots defined by the second pair of distally-extending extension portions.

7. The medical device of claim 6, wherein the at least some of the portion of the first power line extending along the first outer side surface is in a space bounded by: (i) the first outer side surface of the first jaw, (ii) the first extension portion of the first pair of distally-extending extension portions, and (iii) a first extension portion of the second pair of distally-extending extension portions.

8. The medical device of claim 1, wherein the first jaw defines a through-hole through which the first power line extends.

9. The medical device of claim 8, wherein the first power line distally extends along the first outer side surface of the first jaw, then passes through the through-hole defined by the first jaw, and is then electrically coupled to the first electrode.

10. The medical device of claim 1, further comprising a cutter that is proximally and distally movably between the first and second jaws.

11. The medical device of claim 1, wherein a portion of the second power line extends along the second outer side surface.

12. The medical device of claim 11, wherein the portion of the second power line is disposed in a second groove defined along the second outer side surface.

13. The medical device of claim 12, wherein the second power line exits a distal end of the second groove and then passes into a second hole that is defined by the second jaw, and wherein the second power line is electrically coupled to the second electrode in the second hole.

14. The medical device of claim 11, wherein the portion of the first power line that extends along the first outer side surface is rotationally disposed substantially 180° opposite of a central axis of the medical device in comparison to the portion of the second power line extends along the second outer side surface.

15. A medical device comprising:
a first jaw comprising a first electrode;
a second jaw comprising a second electrode,
wherein the first and second jaws are reconfigurable between: (i) a closed state in which the first electrode is adjacent to the second electrode to pinch tissue therebetween and (ii) an opened state in which the first and second electrodes are separated farther from each other than in the closed state;
a first power line electrically coupled to the first electrode; and
a second power line electrically coupled to the second electrode,
wherein the first and second power lines are arranged relative to the first and second jaws such that no additional tensile force is applied to the first and second power lines as the first and second jaws are reconfigured between the opened and closed states.

16. The medical device of claim 15, wherein a portion of the first power line is disposed in a first groove defined along an outer side surface of the first jaw, and wherein a portion of the second power line is disposed in a second groove defined along an outer side surface of the second jaw.

17. The medical device of claim 16, further comprising a rotation shaft about which at least one of the first jaw and the second jaw rotate during reconfiguration between the open and closed states.

18. The medical device of claim 17, wherein at least some of the portion of the first power line that is disposed in the first groove is proximal of the rotation shaft, and wherein at least some of the portion of the second power line that is disposed in the second groove is proximal of the rotation shaft.

19. The medical device of claim 15, wherein the first jaw defines a first through-hole through which the first power line extends, and wherein the second jaw defines a second through-hole through which the second power line extends.

20. The medical device of claim 15, further comprising:
a rotation shaft about which at least one of the first jaw and the second jaw rotate during reconfiguration between the open and closed states;
a first tubular body having a first pair of distally-extending extension portions to which the rotation shaft is mounted; and
a second tubular body slidably disposed within the first tubular body and having a second pair of distally-extending extension portions, wherein the rotation shaft extends through slots defined by the second pair of distally-extending extension portions.
